(19) United States
(12) Patent Application Publication
(10) Pub. No.: US 2013/0281840 A1
(43) Pub. Date: Oct. 24, 2013

(54) DUAL-MODALITY SCANNING SYSTEM FOR DETECTING BREAST CANCER

(71) Applicants: Christopher Leonard Vaughan, Pinelands (ZA); Michael David Evans, Fish Hoek (ZA)

(72) Inventors: Christopher Leonard Vaughan, Pinelands (ZA); Michael David Evans, Fish Hoek (ZA)

(73) Assignee: CAPERAY MEDICAL (PTY) LTD., Cape Town (ZA)

(21) Appl. No.: 13/642,065

(22) PCT Filed: Jun. 3, 2011
(86) PCT No.: PCT/ZA11/00037

§ 371 Date: Dec. 17, 2012

(30) Foreign Application Priority Data
Jun. 3, 2010 (ZA) 2010/03968

(51) Int. Cl. A61B 6/00

(52) U.S. Cl. CPC A61B 6/4417
USPC 600/425

(57) ABSTRACT

This invention relates to a scanning apparatus for imaging the breast that employs both X-ray and ultrasound technologies to enhance the diagnosis of cancer. There is provided a method for acquiring and co-registering the X-ray and ultrasound images in three-dimensional space, so that: the breast is in the same orientation and degree of compression when X-ray and ultrasound images are obtained; both sets of images are acquired simultaneously so as to minimize the time the woman's breast is held stationary; automated breast ultrasound images of the whole breast are acquired in a single scan; both image modalities are acquired in three dimensions; and radiation dose exposure to the woman is minimized. The method incorporates, in a single device, a system for acquiring full-field digital mammography and automated breast ultrasound images, and an algorithm for performing digital tomosynthesis reconstruction using these images.

This invention relates to a scanning apparatus for imaging the breast that employs both X-ray and ultrasound technologies to enhance the diagnosis of cancer. In particular, the present invention provides a method for acquiring and co-registering the X-ray and ultrasound images in three-dimensional (3D) space.

### BACKGROUND TO THE INVENTION

There is strong evidence to support the screening of women for breast cancer where the traditional imaging modality has been mammography which uses two-dimensional X-rays. The sensitivity (percentage of true positives) of X-ray mammography for the detection of breast cancer varies from 75% to 90%, while the specificity (percentage of true negatives) varies from 90% to 95% (Vaughan, 2011). Although digital X-rays significantly increase specificity in comparison with analogue film (Pisano et al., 2005), a major shortcoming of planar X-ray mammography is that it does not do well when the breasts are dense which is often the case for younger women who are less than 50 years of age (Nothacker et al., 2009).

Although the spatial resolution of ultrasound (0.5 to 1.0 mm) is substantially coarser than digital X-rays (50 to 100 μm), ultrasound is better able to differentiate tissues of different densities and has been used as an adjunct to X-ray mammography for more than forty years (Teixidor & Kazam, 1977). In fact, diagnostic breast ultrasound now plays a vitally important role in the detection of cancer in both young and older women (Hooley et al., 2011). Recent studies based on large patient cohorts have shown that hand-held ultrasound (done in addition to X-ray mammography) has resulted in a significant increase in breast cancer detection rate (Berg et al., 2008; Schaefer et al., 2009). Since hand-held ultrasound suffers from repeatability problems, automated breast ultrasound systems have been developed (Kelly et al., 2010).

There is thus a need for a dual-modality system that combines the best of each modality, full-field digital mammography and automated breast ultrasound, in a single device. The ideal functional attributes of such a system to detect breast cancer should include: (1) breast must be in same orientation and degree of compression when X-ray and ultrasound images are obtained (Novak, 1983); (2) both sets of images must be acquired simultaneously so as to minimize the time the woman's breast is held stationary; (3) automated breast ultrasound images of the whole breast must be acquired in a single scan; (4) both image modalities must be acquired in three dimensions (3D); and (5) radiation dose exposure to the woman must be minimized.

In reviewing the prior art in the field of dual-modality imaging (combining X-rays and ultrasound), there are four basic design concepts that have been patented. None of these concepts has yet been implemented in a commercial product although information about some prototypes has been published and is available in the public domain. Furthermore, none of the prior art fulfills all five of the functional attributes identified above. Each of these four design concepts will now be reviewed and their shortcomings highlighted.

In concept one, the X-ray images are captured by a flat panel digital detector located beneath the breast and an ultrasound probe located above the breast (Shmulewitz, 1995; 1996; Richter, 2000; Dines et al., 2003; 2005; Entrekin & Change, 2004). This probe, which is moved under automated control on top of the compressor, is positioned between the X-ray tube and the breast. The shortcomings of this design concept are: the two sets of images must be gathered sequentially, rather than simultaneously, thus increasing the breast compression time; the small size of the ultrasound probe means that multiple scans are required to cover the whole breast, further increasing compression time; no provision is made for capturing X-ray images in 3D; and, because flat-panel detectors suffer from scatter problems, the radiation exposure to the patient is higher than optimal (Irving et al., 2008).

In concept two, which represents a collaboration between the General Electric Company and University of Michigan, the inventors have essentially adapted concept one and added tomosynthesis, a technology that allows multiple X-ray images to be made of the breast (Kapur et al., 2003; 2005; Booi et al., 2007; Sinha et aL, 2007; Goodsitt et al., 2008). Tomosynthesis, in which the X-ray tube is moved through an arc while the breast and flat panel detector remain stationary, enables 3D images of the breast to be reconstructed (Niklason et al., 1999). While concept two thus solves the problem of capturing both imaging modalities in 3D, it still suffers the other shortcomings of concept one, namely sequential (rather than simultaneous) image capture, multiple scans by the ultrasound probe, and high radiation exposure.

Concept three, which emanates from work done at the Fischer Imaging Corporation, is based on a full-field digital mammography system that uses a slot-scanning approach to acquire a planar X-ray image (Tesic et al., 1999; Hussein et al., 2009). Since the X-ray detector moves beneath the breast platform, it is possible to locate the ultrasound probe parallel to the X-ray detector (Besson & Nields, 2005; Suri et al., 2005; 2010). Because the detector and the probe are both beneath the breast platform it is possible to acquire the two images simultaneously, while the slot-scanning geometry reduces scatter and therefore minimizes radiation exposure to the patient (Lease et al., 2002). However, this concept suffers from the shortcoming that the X-ray fan beam is scanned across the breast by a rotating X-ray tube with a fixed collimator slit, which means that tomosynthesis is not possible and the system can therefore only acquire 2D rather than 3D images.

Concept four, recently patented by the General Electric Company, requires the patient to lie on her stomach in a prone position with her breast protruding through an opening in the horizontal support (Li et al., 2010). Both the X-ray and ultrasound acquisition systems are located beneath the support and rotate around the breast, enabling the capture of 3D images in both modalities. While this concept has the advantage of not requiring compression of the breast, it suffers from two shortcomings: part of the breast tissue, particularly that in the region of the axilla (armpit) or close to the chest wall which is susceptible to invasive cancer, cannot be imaged because the breast does not protrude far enough through the opening in the horizontal support; and the method of acquiring 3D X-ray images, which is based on computer tomography, exposes the patient to an unnecessarily high radiation dose.

It is an object of the invention to provide an alternative scanning apparatus, utilizing both X-ray and ultrasound that addresses all of the shortcomings of the four design concepts described above and meets all five of the functional attributes: (1) breast is in same orientation and degree of compression when X-ray and ultrasound images are obtained; (2) both sets of images are acquired simultaneously so as to minimize the time the woman's breast is held stationary; (3) automated breast ultrasound images of the whole breast are acquired in a single scan; (4) both image modalities are acquired in three dimensions (3D); and (5) radiation dose exposure to the woman is minimized.

### SUMMARY OF THE INVENTION

According to the invention there is provided a dual-modality scanning apparatus comprising:
an X-ray source arranged to generate an output beam having a reference axis;
a pre-collimator arranged to modify the output beam to generate a fan beam;
a platform defining a first surface for supporting a breast of a subject;
a first drive arranged to move the pre-collimator transverse to the reference axis, thereby to impart motion to the fan beam;
a linear scanning element comprising an X-ray sensor and an ultrasound transducer supported adjacent to and beneath the first surface and extending parallel to the fan beam;
a second drive arranged to move the linear scanning element transverse to the reference axis, in synchronization with the first drive, thereby to generate a two-dimensional X-ray image and three-dimensional ultrasound images;
a third drive arranged to rotate the X-ray source relative to the breast platform, thereby to generate a plurality of sets of two-dimensional X-ray images of the breast for tomosynthesis purposes; and
a processor for implementing tomosynthesis reconstruction algorithms to generate three-dimensional X-ray and ultrasound images, and to co-register the three-dimensional X-ray and ultrasound images.

The apparatus may include a fourth drive arranged to move a compression plate so as to compress the breast of the subject against the first surface of the platform.

The apparatus may further include a fifth drive arranged to rotate the whole imaging system so as to accommodate the different views (e.g. cranio-caudal and medio-lateral oblique) of the patient;

The apparatus may include a sixth drive arranged to vary the height of the imaging system relative to a support.

The X-ray source is preferably arranged to generate an output beam in the form of a cone beam from which the pre-collimator generates the fan beam.

Preferably the pre-collimator defines a slot that can be moved between each of a plurality of selected positions to generate respective fan beams. More preferably, the slot is moved via the first drive.

The pre-collimator is preferably arranged to be moved continuously through a range of selected positions.

The first and second drives are preferably arranged to be operated in synchronization, so that for each position of the X-ray source and the linear scanning element, a fan beam is generated which coincides substantially with the position of the X-ray sensor.

The third drive is preferably arranged to enable the X-ray source, including the pre-collimator slot, to be rotated relative to the breast platform.

The fourth drive is preferably arranged to provide compression to the breast, which compression can be rapidly released upon completion of image capture.

The fifth drive is preferably arranged to permit isocentric rotation of the imaging system to be rotated between the cranio-caudal and medio-lateral oblique positions without substantial movement of the patient.

The sixth drive is preferably arranged to provide vertical movement of the imaging system relative to a support to accommodate patients of varying heights.

The apparatus preferably includes a post-collimator located adjacent to the first surface of the platform and defining a slot through which the respective fan beams can pass to reach the X-ray sensor.

The platform preferably has a width substantially equal to the width of the largest breast to be measured.

The apparatus preferably includes a controller arranged to operate the respective drives, the X-ray source and the ultrasound transducer, to carry out simultaneous capture of X-ray and ultrasound image data.

The processor preferably includes a tomosynthesis reconstruction algorithm that takes advantage of the multiple three-dimensional ultrasound images to enhance the spatial resolution of this imaging modality.

### BRIEF DESCRIPTION OF THE DRAWINGS

FIG. 1 is a pictorial view of scanning apparatus according to an embodiment of the invention;

FIG. 2 is a pictorial view of scanning apparatus, illustrating the generation of X-ray fan beams at each of a plurality of different positions relative to a reference axis;

FIG. 3 is a pictorial view of scanning apparatus, illustrating the angular rotations required to generate three different sets of breast images; and

FIG. 4 is a block schematic diagram showing major components of the apparatus.

### DESCRIPTION OF PREFERRED EMBODIMENTS

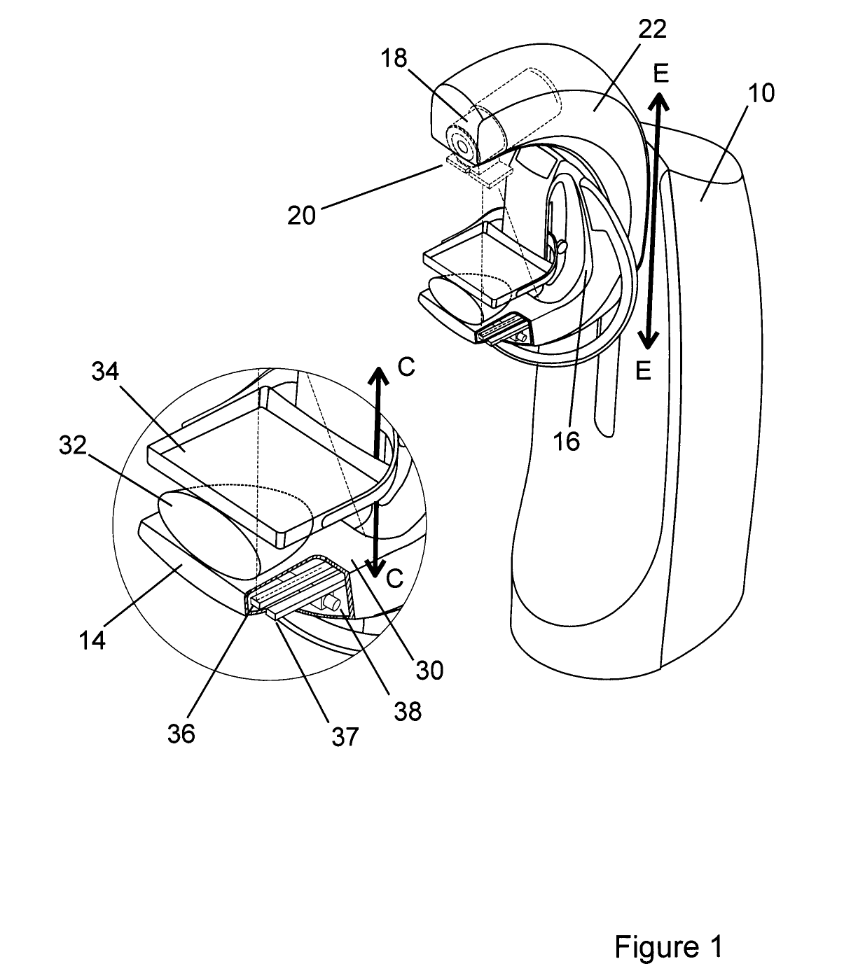

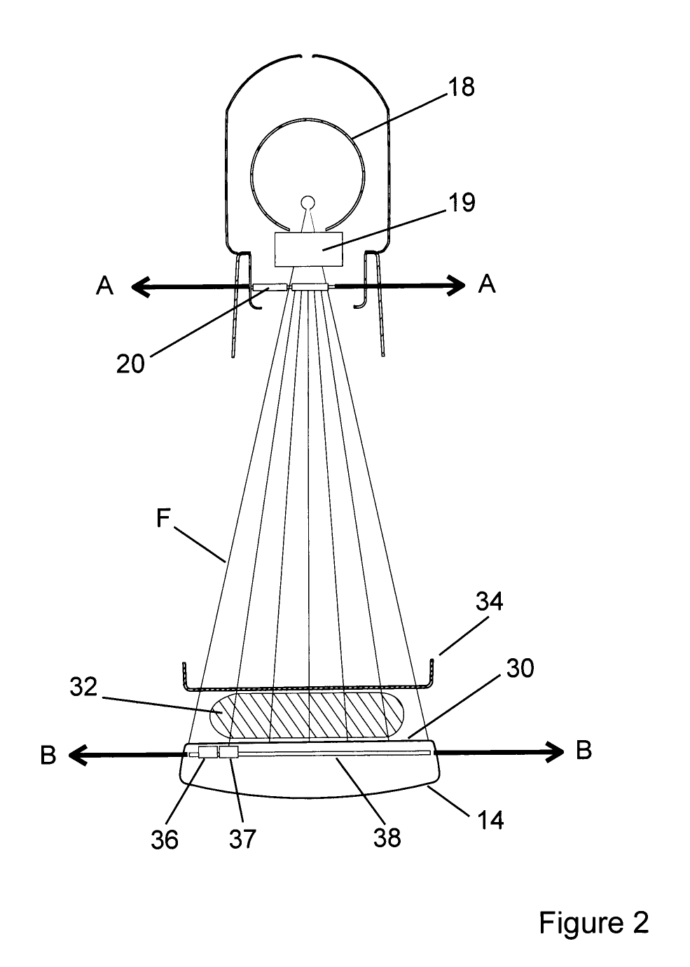

FIGS. 1 and 2 are pictorial illustrations of an example embodiment of the dual-modality scanning apparatus according to the present invention. The apparatus includes a support pillar 10 on which is mounted a C-arm. The C-arm includes a breast support platform 14, an upright member 16 mounted rotatable to the pillar 10, a further upright member 22 mounted rotatable to the upright member 16, and an X-ray source 18 with an associated beam-shaper 19 and pre-collimator 20, attached to the upright member 22, which extends parallel to the platform 14.

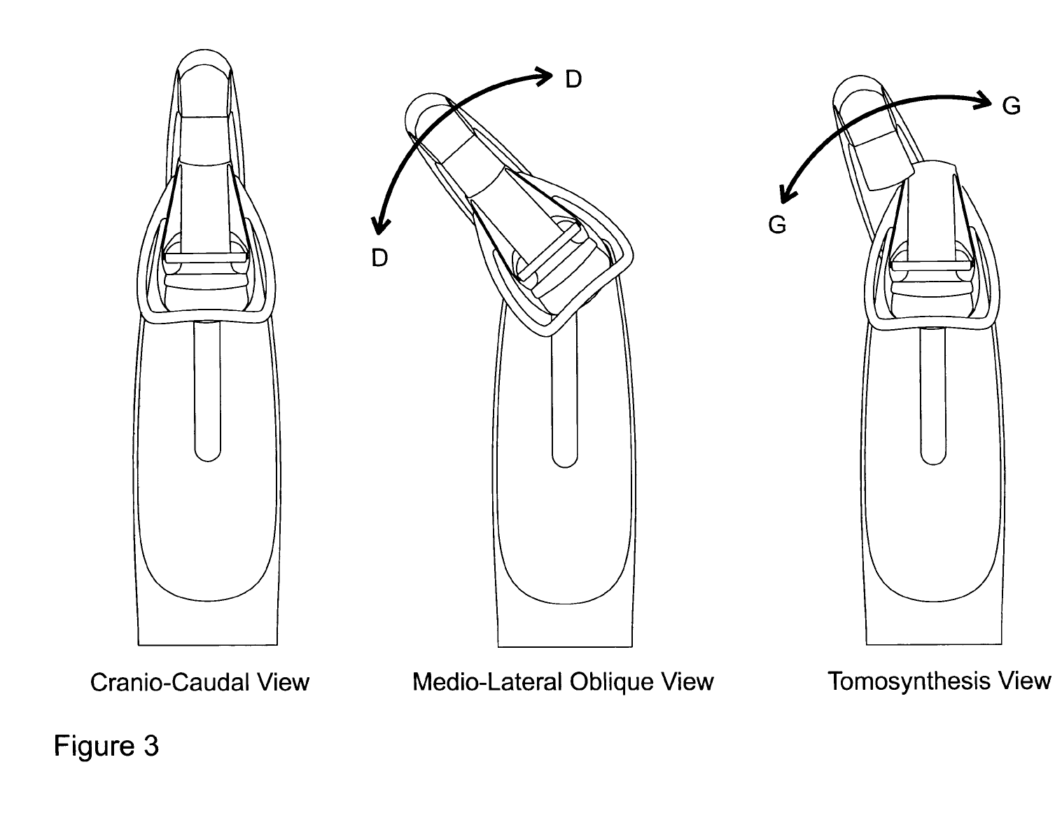

The entire C-arm can be moved up and down in the direction of the arrows E and rotated about its attachment point on the pillar 10 about an axis of rotation, as indicated by the arrows D in FIG. 3, by respective drives.

The breast support platform 14 defines a breast support surface 30 on which a breast 32 of a human subject can be placed. A breast compressor plate 34 with an associated clamp mechanism is positioned adjacent to the breast support surface.

The breast compressor has its own linear drive and can be adjusted in the direction of the arrows C to compress the breast 32 firmly against the breast support surface 30.

The dimensions of the breast support platform 14 are chosen to be as narrow as possible, consistent with the width of the largest breast to be measured. This allows positioning of the subject's breast over an outer corner of the platform, thus enabling the breast compression plate 34 to draw the subject's axilla tissue into the scanner's field of view. This ensures that the tissue in the subject's axillary breast region is fully imaged. Conventional imaging systems having a wide breast support platform cannot easily achieve this.

Below the breast support surface 30 (which is defined by a material that is both transparent to X-rays and has low acoustical impedance, such as Polymethylpentene, which is also known as TPX) is a linear slot X-ray detector assembly 36 and an ultrasound transducer assembly 37 which are mounted on a pair of rails 38 for transverse movement in a plane parallel to the breast support surface and parallel to a plane defined by the transverse movement of the X-ray source 18. This is indicated by the arrows B. Thus, the linear slot X-ray detector and the ultrasound transducer together define a dual-modality linear scanning element, enabling simultaneous acquisition of X-ray and ultrasound images.

The X-ray source 18 has an associated beam shaper 19 that generates a cone beam of X-rays that diverges outwardly towards the platform 14, with a diameter sufficient to ensure coverage of a breast 32 to be imaged by the resultant fan beams. The cone beam has a central upright reference axis that is normal to the plane defined by the breast support surface 30 and the planes in which the X-ray source and the linear X-ray slot detector assembly 36 move. Typically, the cone beam has a cone angle of approximately 30 degrees.

The pre-collimator 20 defines a linear slot which, when placed in the path of the X-ray cone beam, generates a generally planar fan beam which is narrowest adjacent to the pre-collimator and which broadens in the direction of the platform 14. The pre-collimator 20 can be moved in the direction of the arrows A in a plane parallel to the breast support surface 30 so that a number of fan beams F, each having a different inclination relative to the upright reference axis of the X-ray cone beam, can be generated, according to the orientation of the pre-collimator slot. This is indicated schematically in FIG. 2. Thus, each fan beam is inclined at a different pre-determined angle relative to the reference axis of the X-ray cone beam.

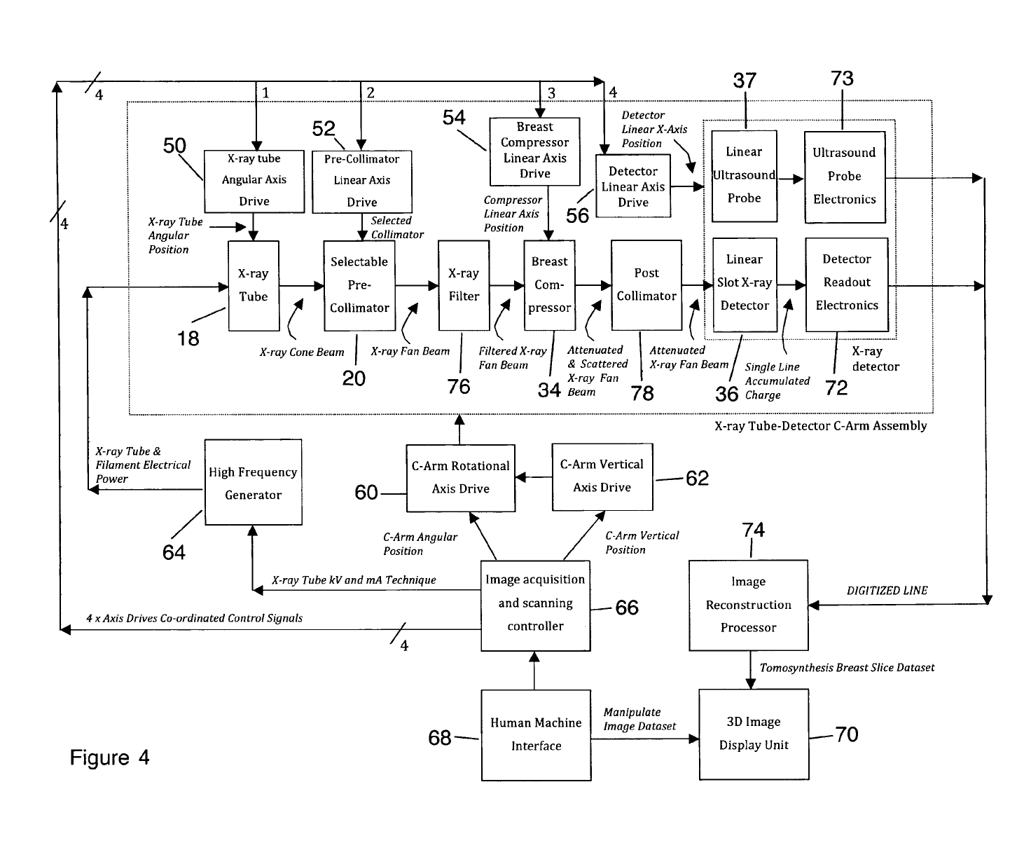

The pre-collimator can be arranged to be indexed between predetermined positions, but is preferably driven continuously between desired positions which are selected according to the number of fan beams required and the size of the breast to be scanned. With reference now also to FIG. 4, a post-collimator 78 is located between the breast support surface 30 of the platform 14 and the detector 36. The post-collimator will typically comprise a layer of lead sandwiched between layers of aluminium. The post-collimator has slots with a width calculated to eliminate any penumbra from the impinging fan beams, typically having a width of about 4 mm for a pre-collimator slot width of 0.4 mm.

The upright member 22, to which is attached the X-ray source 18, can be uncoupled from the upright member 16, and rotated in the direction of the arrows G, thus allowing a plurality of X-ray images of the breast 32 to be captured from the different angles (see FIG. 3). These multiple two-dimensional images can be used to generate a three-dimensional image of the breast using a digital tomosynthesis algorithm.

The linear scanning element used in the prototype embodiment of the apparatus was a CCD based detector of approximately 6 millimetre width, comprising 128 rows of pixels, each with a 48 micron width. This, in conjunction with a fan beam of 3 to 5 millimetres width, permits operation of the CCD detector in a Time Delay and Integration (TDI) mode which provides superior sensitivity and lower noise, enabling the use of a lower X-ray dose than would otherwise be required. It is expected that a CMOS detector will allow faster data clocking and consequently a higher scan speed of approximately 150 mm/second compared to the CCD detector.

FIG. 4 is a schematic block diagram showing major components of the apparatus. The pre-collimator 20 has an associated linear axis drive 52 which adjusts the position of the pre-collimator slot in order to generate an X-ray fan beam at the required angle. The breast compressor plate 34 has an associated linear axis drive 54, and the X-ray detector 36 and ultrasound transducer 37 have an associated linear axis drive 56 that moves the detector 36 and the transducer 37 transversely in the platform 14 below the breast support surface 30.

The C-arm has two associated drives, a rotational axis drive 60 which permits rotation of the C-arm on the pillar 10, and a vertical axis drive 62 which allows adjustment of the vertical position of the C-arm on the pillar. High precision 25 linear position encoders associated with the pre-collimator and the slot detector drives provide the alignment control position data required for the fan beam to be incident on the slot detector's imaging element. The apparatus is assembled in such a way that the linear guides of the X-ray source, pre-collimator and slot detector linear axes are precisely parallel and the slot detector itself is precisely parallel to the fan beam (that is, orthogonal to its linear axis).

The X-ray source or tube 18 is powered by a high frequency generator 64 that is controlled by an image acquisition and scanning controller 66. The controller 66 also controls the respective linear axis drives, and the rotational and vertical C-arm drives 60 and 62. The controller 66 is connected to a human machine interface 68, typically a computer terminal, with an associated 3D image display unit 70. The X-ray detector 36 has associated readout electronics 72 integrated with it, and the ultrasound transducer 37 has associated electronics 73 integrated with it, both of which feed raw data to an image reconstruction processor 74.

The operation of the apparatus will now be described in greater detail. Prior to an imaging examination the C-arm is rotated about its axis of rotation on the pillar 10 to the desired angle, between 0 and 180 degrees, and driven to the correct vertical height on the pillar to match the subject's breast height. The three linear axes of the respective drives on the C-arm for the pre-collimator 20, the breast compressor plate 14, and the detector assembly 36 are synchronized and all moved to their home positions.

The subject's breast 32 is then placed on the breast support surface 30 of the platform 14 and the breast compressor plate 34 is activated to provide a preliminary compressive pressure, to immobilize the breast and pull breast tissue away from the chest wall. A start signal is then issued via the human machine interface 68 to initiate a breast scan.

An extra compressive force is then applied by the drive of the breast compressor plate 34. The X-ray source 18 is energized by the high frequency generator 64, with a preliminary exposure technique (kV & mA) based on the breast thickness, implied from the breast compressor linear position, to generate an X-ray cone beam which is pre-collimated by the pre-collimator 20 and filtered by the X-ray filter 76 to produce a filtered X-ray fan beam F.

Tight collimation (0.4 mm) near the source minimizes the back scatter to the subject when compared with collimation techniques applied on top of the breast. With an X-ray source focal spot size of 0.3 mm and a source to detector distance of 650 mm, cone beam collimation using a slot with a width of 0.4 mm results in a primary fan beam width of approximately 4 mm incident on the detector 36, which has an active width of 6 mm. This provides significantly greater X-ray flux and better tube loading characteristics when compared with slit scanning configurations that collimate to the width of single pixel detectors 0.1 mm wide.

The pre-collimator 20 moves in synchronization with the X-ray detector 36 to cause the X-ray fan beam to move across the breast at a linear velocity of up to 150 mm/s. The relative positioning of the X-ray source 18, the pre-collimator 20, and the X-ray detector 36 to achieve correct beam alignment is performed according to the calibration data acquired from an automatic beam alignment process. The filtered X-ray fan beam is attenuated and scattered by the breast. The X-ray fan beam post-collimator 78 collimates the X-ray photon flux exiting the breast to eliminate the scattered photons and ensure that only the primary photons impinge on the X-ray slot detector 36.

The multi row linear slot X-ray detector 36 operates in Time Delay and Integration (TDI) mode and the detector readout line clock is electronically synchronized with the detector linear axis velocity. The detector accumulates charge across each row of pixels in the imaging element to provide a single image line to an analogue-to-digital convertor in the readout electronics 72. Digitized line data is compiled into a two-dimensional image projection during the scan by the image reconstruction processor 74.

The linear ultrasound transducer 37, together with its associated control electronics 73, generates two-dimensional images for each position of the transducer. Because the transducer 37 moves in synchrony with the X-ray detector 36 along the pair of guide rails 38 and 40, it is able to capture multiple 2D images which, when fed into the image reconstruction processor 74, generates 3D ultrasound images of the breast 32. The processor 74 is also able to co-register the 2D X-ray image with the 3D ultrasound image, thus aiding in the diagnosis of underlying pathology.

The apparatus can also be used to gather multiple 2D X-ray images of the breast. This is accomplished by rotating the X-ray tube 18 in the direction of the arrows G by means of the angular axis drive 50, and repeating the X-ray image 10 acquisition sequence described previously. The multiple 2D images can then be sent to the image reconstruction processor 74 which then implements a digital tomosynthesis algorithm to generate 3D X-ray images of the breast. These 3D X-ray images can be co-registered with the 3D ultrasound images to improve diagnosis.

The above described example embodiment of scanning apparatus according to the invention has a number of advantages compared with known apparatus. By locating an ultrasound transducer in parallel with a linear slot scanning X-ray detector, it allows for the simultaneous capture of X-ray and ultrasound images, thus improving the sensitivity of the apparatus significantly. The use of a narrow breast support platform allows positioning of a subject's breast over the platform corner, enabling the compressor plate to draw the subject's axillary tissue into the scanner's field of view.

The use of a slot detector that utilizes TDI techniques to optimise signal-to-noise ratio and reduce scatter is also advantageous, allowing clear imaging at low X-ray doses. A suitable CMOS detector, in particular, is relatively inexpensive compared with photon counter detectors used in known apparatus. The use of a CMOS detector is also desirable as its high speed (relative to CCD circuitry) allows fast data clocking and thus scan speeds, typically 150 mm/s. This enables the collection of a full data set of a large breast (250 mm wide) in less than two seconds. The scan duration is minimized by edge detection of the breast under examination, limiting the scan travel to the edge extent of the breast.

### REFERENCES

Berg W A, Blume J D, Cormack J B, Mendelson E B, Lehrer D, Böhm-Vélez M, Pisano E D, Jong R A, Evans W P, Morton M J, Mahoney M C, Larsen L H, Barr R G, Farria D M, Marques H S, Boparai K, and other ACRIN 6666 Investigators, “Combined screening with ultrasound and mammography vs mammography alone in women at elevated risk of breast cancer”, Journal of the American Medical Association, 299(18): 2151-2163, 2008.
Besson G M, Nields M W, “Integrated X-ray and ultrasound medical imaging system”, U.S. Pat. No. 6,846,289, 25 Jan. 2005.
Booi R C, Krücker J F, Goodsitt M M, O'Donnell M, Kapur A, LeCarpentier G L, Roubidoux M A, Fowlkes J B, Carson P L, “Evaluating thin compression paddles for mammographically compatible ultrasound”, Ultrasound in Medicine and Biology, 33(3): 462-482, 2007.
Dines K A, Kelly-Fry E, Romilly A P, “Mammography method and apparatus”, U.S. Pat. No. 6,574,499, 3 Jun. 2003.
Dines K A, Kelly-Fry E, Romilly A P, “Mammography method and apparatus”, U.S. Pat. No. 6,876,879, 5 Apr. 2005.
Entrekin R R, Change N D, “Compression plate for diagnostic breast imaging”, U.S. Pat. No. 6,682,484, 27 Jan. 2004.
Goodsitt M M, Chan H P, Hadjiiski L, LeCarpentier G L, Carson P L, “Automated registration of volumes of interest for a combined x-ray tomosynthesis and ultrasound breast imaging system”, Digital Mammography, E A Krupinski (Editor), Lecture Notes in Computer Science 5116, Springer-Verlag, Berlin, pp. 463-468, 2008.
Hooley R J, Andrejeva L, Scoutt L M, “Breast cancer screening and problem solving using mammography, ultrasound, and magnetic resonance imaging”, Ultrasound Quarterly, 27(1): 23-47, 2011.
Hussein K, Vaughan C L, Douglas T S, “Modeling, validation and application of a mathematical tissue-equivalent breast phantom for linear slot-scanning digital mammography”, Physics in Medicine and Biology, 54(6): 1533-1553, 2009.
Irving B J, Maree G J, Hering E R, Douglas T S, “Radiation dose from a linear slit scanning x-ray machine with full-body imaging capabilities”, Radiation Protection Dosimetry, 130(4): 482-489, 2008.
Kapur A, Eberhard J W, Yamrom B, Themenius K E, Buckley D J, Johnson R N, Wirth R F, Astley O, Opsahl-Ong B, Muller S L W, Karr S, “Method, system and apparatus for digital imaging”, United States Patent Application Number US 2003/0149364 A1, 7 Aug. 2003.
Kapur A, Thomenius K E, “Acoustic coupling gel for combined mammography and ultrasound image acquisition and methods thereof”, United States Patent Application Number US 2005/0288581 A1, 29 Dec. 2005.
Kelly K M, Dean J, Comulada W S, Lee S-J, “Breast cancer detection using automated whole breast ultrasound and mammography in radiographically dense breasts”, European Radiology, 20: 734-742, 2010.
Lease A, Vaughan C, Beningfield S, Potgieter H, Booysen A, “Feasibility of using LODOX technology for mammography”, Medical Imaging 2002: Physics of Medical Imaging, Edited by L E Antonuk and M J Yaffe, Proceedings of SPIE, Bellingham, W A, Vol. 4682, pp. 656-664, 2002.
Li B, Thibault J B, Hall A L, “Combining X-ray and ultrasound imaging for enhanced mammography”, U.S. Pat. No. 7,831,015, 9 Nov. 2010.
Niklason L T, Niklason L E, Kopans D B, “Tomosynthesis system for breast imaging”, U.S. Pat. No. 5,872,828, 16 Feb. 1999.
Nothacker M, Duda V, Hahn M, Warm M, Degenhardt F, Madjar H, Weinbrenner S, Albert U S, “Early detection of breast cancer: benefits and risks of supplemental breast ultrasound in asymptomatic women with mammographically dense breast tissue. A systematic review”, BMC Cancer, 9:335, doi: 10.1186/1471-2407-9-335, 2009.
Novak D, “Indications for and comparative diagnostic value of combined ultrasound and X-ray mammography”, European Journal of Radiology, (Supplement 1): 299-302, 1983.
Pisano E D, Gatsonis C, Hendrick E, Yaffe M, Baum J K, Acharyya S, Conant E F, Fajardo L L, Bassett L, D'Orsi C, Jong R, Rebner M and the Digital Mammographic Imaging Screening Trial (DMIST) Investigators Group, “Diagnostic performance of digital versus film mammography for breast-cancer screening”, New England Journal of Medicine, 353(17): 1773-1783, 2005.
Richter K, “Combined ultrasound and X-ray device for breast examination”, German Patent Number DE 198 37 264, 9 Mar. 2000.
Schaefer F K W, Waldmann A, Katalinic A, Wefelnberg C, Heller M, Jonat W, Schreer I, “Influence of additional breast ultrasound on cancer detection in a cohort study for quality assurance in breast diagnosis—analysis of 102,577 diagnostic procedures”, European Radiology, doi: 10.1007/s00330-009-1641-x, 2009.
Shmulewitz A, “Methods and apparatus for performing sonomammography”, U.S. Pat. No. 5,474,072, 12 Dec. 1995.
Shmulewitz A, “Methods and apparatus for performing sonomammography and enhanced x-ray imaging”, U.S. Pat. No. 5,479,927, 2 Jan. 1996.
Sinha S P, Roubidoux M A, Helvie M A, Nees A V, Goodsitt M M, LeCarpentier G L, Fowlkes J B, Chalek C L, Carson P L, “Multi-modality 3D breast imaging with X-ray tomosynthesis and automated ultrasound”, Proceedings of the 29th International Conference of the IEEE EMBS, Lyon, France, pp. 1335-1338, 23-26 Aug. 2007.

1. A dual-modality scanning apparatus comprising:
an X-ray source arranged to generate an output beam having a reference axis;
a pre-collimator arranged to modify the output beam to generate a fan beam;
a platform defining a first surface for supporting a breast of a subject;
a first drive arranged to move the pre-collimator transverse to the reference axis, thereby to impart motion to the fan beam;
a linear scanning element comprising an X-ray sensor and an ultrasound transducer supported adjacent to and beneath the first surface and extending parallel to the fan beam;
a second drive arranged to move the linear scanning element transverse to the reference axis, in synchronization with the first drive, thereby to generate a two-dimensional X-ray image and three-dimensional ultrasound images;
a third drive arranged to rotate the X-ray source relative to the breast platform, thereby to generate a plurality of sets of two-dimensional X-ray images of the breast for tomosynthesis purposes; and
a processor for implementing tomosynthesis reconstruction algorithms to generate three-dimensional X-ray and ultrasound images, and to co-register the three-dimensional X-ray and ultrasound images.

2. A dual-modality scanning apparatus according to claim 1 wherein a fourth drive is arranged to move a compression plate so as to compress the breast of the subject against the first surface of the platform.

3. A dual-modality scanning apparatus according to claim 2 wherein a fifth drive is arranged to rotate the whole imaging system so as to accommodate the different views of the patient.

4. A dual-modality scanning apparatus according to claim 3 wherein a sixth drive is arranged to vary the height of the imaging system relative to support.

5. A dual-modality scanning apparatus according to claim 1 wherein the X-ray source is arranged to generate an output beam in the form of a cone beam from which the pre-collimator generates the fan beam.

6. A dual-modality scanning apparatus according to claim 5 wherein the pre-collimator defines a slot that can be moved between each of a plurality of selected positions via the first drive to generate respective fan beams.

7. A dual-modality scanning apparatus according to claim 6 wherein the pre-collimator is arranged to be moved continuously via the first drive through a range of selected positions.

8. A dual-modality scanning apparatus according to claim 7 wherein the first and second drives are arranged to be operated in synchronization, so that for each position of the X-ray source and the linear scanning element, a fan beam is generated which coincides substantially with the position of the X-ray sensor.

9. A dual-modality scanning apparatus according to claim 8 wherein the third drive is arranged to enable the X-ray source, including the pre-collimator slot, to be rotated relative to the breast platform.

10. A dual-modality scanning apparatus according to claim 2, wherein the fourth drive is arranged to provide compression to the breast, which compression can be rapidly released upon completion of image capture.

11. A dual-modality scanning apparatus according to claim 3, wherein the fifth drive is arranged to permit isocentric rotation of the imaging system to be rotated between the cranio-caudal and medio-lateral oblique positions without substantial movement of the patient.

12. A dual-modality scanning apparatus according to claim 4, wherein the sixth drive is arranged to provide vertical movement of the imaging system relative to a support to accommodate patients of varying heights.

13. A dual-modality scanning apparatus according to claim 6, wherein the apparatus includes a post-collimator located adjacent to the first surface of the platform and defining a slot through which the respective fan beams can pass to reach the X-ray sensor.

14. A dual-modality scanning apparatus according to claim 1, wherein the platform has a width substantially equal to the width of the largest breast to be measured.

15. A dual-modality scanning apparatus according to claim 1, wherein the apparatus includes a controller arranged to operate the respective drives, the X-ray source and the ultrasound transducer, to carry out simultaneous capture of X-ray and ultrasound image data.

16. A dual-modality scanning apparatus according to claim 1, wherein the apparatus includes a processor arranged to implement a tomosynthesis reconstruction algorithm that takes advantage of the multiple three-dimensional ultrasound images to enhance the spatial resolution of this imaging modality.
